(19) United States
(12) Patent Application Publication
(10) Pub. No.: US 2024/0084300 A1
(43) Pub. Date: Mar. 14, 2024

(54) MIRNA COMPOSITION COMPRISING 11 SPECIFIC MIRNAS AND ITS USE IN THE TREATMENT OF CANCER

(71) Applicants: INSERM (INSTITUT NATIONAL DE LA SANTE ET DE LA RECHERCHE MEDICALE), Paris (FR); UNIVERSITÉ DE PARIS, Paris 6 (FR)

(72) Inventor: Sébastien JAULIAC, CLICHY (FR)

(21) Appl. No.: 18/268,450

(22) PCT Filed: Dec. 20, 2021
(86) PCT No.: PCT/EP2021/086712

§ 371 Date: Jun. 20, 2023

(30) Foreign Application Priority Data
Dec. 21, 2020 (EP) 20306636.0

(51) Int. Cl. C12N 15/113; A61K 9/50; A61P 35/00

(52) U.S. Cl. CPC C12N 15/113; A61K 9/5068; A61P 35/00; C12N 2310/141; C12N 2320/32

(57) ABSTRACT

The present invention relates to a miRNA composition comprising the following 11 miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, and hsa-miR-7704. Optionally, it may further comprise at least one miRNA selected from hsa-miR-195-5p, hsa-miR-324-3p, hsa-miR-4449 and hsa-miR-501-5p. It further relates to liposomes or SEVs loaded by said miRNA composition, to methods for preparing the loaded SEVs, to a pharmaceutical composition comprising the miRNA composition, liposomes or SEVs, and to therapeutic uses of the pharmaceutical composition, in particular in the treatment of cancer.

### TECHNICAL FIELD OF THE INVENTION

The present invention is in the field of oncology, and more particularly of the treatment of cancer and metastatic cancer. It relates to a miRNA composition comprising the following 11 miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, and hsa-miR-7704. Optionally, it may further comprise at least one miRNA selected from hsa-miR-195-5p, hsa-miR-324-3p, hsa-miR-4449 and hsa-miR-501-5p. It further relates to liposomes or SEVs loaded by said miRNA composition, to methods for preparing the loaded SEVs, to a pharmaceutical composition comprising the miRNA composition, liposomes or SEVs, and to therapeutic uses of the pharmaceutical composition, in particular in the treatment of cancer.

### BACKGROUND ART

Breast cancer is a leading cause of morbidity in women worldwide. The main reason of death for these patients is not the primary tumor, but distant metastases, which are directly linked to the migratory and invasive phenotype of the cancer cells. In addition, de novo and acquired resistance to anticancer agents remains a major obstacle in the treatment of breast cancer. Moreover, resistance to treatment is often associated to increased invasiveness of cancer and development of metastasis, suggesting that some treatment, while initially efficient to treat the primary cancer, at the same time promote the appearance of more invasive cells, finally resulting in metastasis combined to resistance to treatment. The same is true for other cancers. There is thus a need for new therapies that would be efficient not only on the primary cancer but that would also inhibit the development of invasiveness and metastasis.

Moreover, it is now well known that no therapy is efficient in all patients, a varying proportion of patients being resistant to any given therapy. In order to prevent long-term administration of a non-efficient treatment (which is not only costly but also detrimental to the patient's health), there is also a need for tests with the ability to predict or at least to determine efficiency of a given therapy for a given patient, before or just after treating the patient.

Different studies revealed that secreted extracellular vesicles (SEV) can be readily detected in tumor tissue and many body fluids, and are found in higher concentrations, both in tumor tissue, and in the serum and plasma of cancer patients (Tickner et al. Front Oncol 4, 127 (2014)).

Secreted extracellular vesicles (SEV), is the general term to designate cell secreted vesicles ranging approximately 40nm to few pm in size. Among them, exosomes comprise the most prominently described classes of SEV. Exosomes have a diameter lower than about 150 nm and are derivatives of the endosomal compartment. SEV contain cytosolic and membrane proteins derived from the parental cells. The protein content of SEV depends on their cellular origin and SEV are enriched for certain molecules, especially endosome-associated proteins (e.g. CD63) and proteins involved in multivesicular bodies formation, but also contain targeting/adhesion molecules. Remarkably, SEV contain not only proteins but also functional mRNAs, long non-coding RNAs and miRNAs, and in some cases, they have been shown to deliver these genetic materials to recipient cells.

The cargo of SEV is potentially particularly interesting for targeting therapies to tumors, as SEV are secreted in the extracellular compartment, where their content is protected from degradation because of their lipid membrane, and SEV excreted from one cell are known to be able to fuse with surrounding cells, and thus have the potential to initiate signaling responses (Hendrix, A. et al. Cancer Res. 70, 9533-9537 (2010)).

However, preliminary studies rather suggested that cancer cells-derived SEV were rather deleterious than helpful for cancer treatment, being involved in cancer progression and metastasis (van der Pol E. et al. Pharmacol Rev 64:A-AD, 2012; Yu et al. Cancer Sci 106 (2015) 959-964; Peinado, H. et al. Nat. Med. 18, 883-891 (2012); Takahashi, Y. et al. J. Biotechnol. 165, 77-84 (2013); Melo, S. A. et al. Cancer Cell 26, 707-721 (2014)).

More recently, after it had been shown that NFAT3 is specifically expressed in estrogen receptor a positive (ERA+) breast cancer cells of low invasive capacity, and that transduction with a vector of expression of NFAT3 inhibits invasion of both ERA+ (low invasive capacity) and ERA− (high invasive capacity) breast cancer cells (Fougère, M., et al. (2010). Oncogene, 29(15), 2292-2301), it was further found that SEVs derived from luminal breast cancer cell lines expressing endogenously NFAT3 were inhibitory, being fully competent to impair triple negative breast cancer (TNBC) cell lines invasion but interestingly also the invasion of other highly aggressive cancers (melanoma, glioblastoma and pancreatic cancer) in vitro. Critically, these in vitro results were expanded in an in vivo orthotopic TNBC mice model where these inhibitory SEVs were qualified to inhibit metastases arising and tumor growth. Moreover, these SEVs were efficient to block the growth of a pre-established tumor. Mechanistically, expression of a functional NFAT3 in the SEVs-producing cells was proven to be necessary for SEVs to exert their inhibitory functions both in vitro (inhibition of cell invasion and hetero spheroids growth) and in vivo (inhibition of tumor growth and metastases arising). With the idea in minds to develop efficient tools to treat aggressive cancers, we established the proof of concept that over-expressing a constitutively active form of NFAT3 in the SEVs-producing cells was a suitable way to increase the inhibitory function of the resulting SEVs in vitro and in vivo (WO2017167788A1; de Camargo, L. C. B et al. Sci Rep 10, 8964 (2020)).

However, the components of the SEVs that were responsible for the anticancer activity were not identified among the numerous types of compounds included in the SEVs. In addition, using SEVs derived from cancer cells may raise regulatory issues. There is thus a need for further anticancer therapies.

### SUMMARY OF THE INVENTION

As explained above, SEVs contain many types of compounds, including various proteins (the content of which varies depending on the producing cell), but also various types of RNAs, such as functional mRNAs, long non-coding RNAs and miRNAs. Identifying which component of the complex content of SEVs derived from cells overexpressing NFAT3 was thus a complex task. In the context of the present invention, the inventors surprisingly found that several miRNAs contained in SEVs derived from cells overexpressing NFAT3 were involved and able to reproduce in the anticancer activity of the SEVs, a specific combination of 11 miRNAs being able to inhibit cancer cells motility, and 8 miRNAs being able to inhibit the growth of cancer cells/macrophages hetero-spheroids.

In a first aspect, the present invention thus relates to a miRNA composition comprising the following 11 miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, and hsa-miR-7704. Said miRNA composition is able to inhibit cancer cells motility, and thus has antimetastatic properties. Moreover, 4 of the 11 miRNAs of the composition also have the ability to inhibit the growth of cancer cells/macrophages hetero-spheroids and thus have general anticancer properties.

The 11 miRNAs are preferably present in the composition in specific relative ratios.

Moreover, the miRNA composition may preferably further comprise at least one of the 4 other mRNAs identified by the inventors as being able to inhibit the growth of cancer cells/macrophages hetero-spheroids, i.e. at least one miRNA selected from hsa-miR-195-5p, hsa-miR-324-3p, hsa-miR-4449 and hsa-miR-501-5p.

For practical reasons, the miRNA composition preferably comprises at most 50 distinct miRNAs.

In a second aspect, the present invention also relates to a delivery vector containing the miRNA composition according to the invention, more preferably liposomes or SEVs loaded with the miRNA composition according to the invention.

In a third aspect, the present invention also relates to a method for preparing the loaded SEVs according to the invention, comprising:
a) preparing SEVs from healthy cells,
b) loading the SEVs with a miRNA composition according to the invention.

In a fourth aspect, the present invention also relates to a pharmaceutical composition comprising the miRNA composition, the liposome, or the SEV according to the invention or prepared using the method according to the invention or mixtures thereof, for use as a medicament.

In a fifth aspect, the present invention also relates to a pharmaceutical composition comprising the miRNA composition, the delivery vector (in particular the liposome, or the SEV according to the invention or prepared using the method according to according to the invention) or mixtures thereof, for use in the treatment of cancer. The treated cancer is preferably selected from solid cancers; more preferably from breast cancer, melanoma, pancreatic cancer, glioblastoma, colorectal cancer, and lung cancer, and most preferably is breast cancer. Said cancer may be non-aggressive or aggressive primary cancer, or even metastatic cancer.

### DESCRIPTION OF THE FIGURES

FIG. 1. The anti-invasive miRNAs combination inhibits cell invasion as EVs does. MDA-MB-231 cells were transfected with either 25 nM of the miRNA control or 25 nM of the anti-invasive miRNAs combination. The following day, cells were treated or not with EVs and subjected to an invasion assay (n=3, ***p<0.001).

FIG. 2. The combination of the 15 miRNAs (miRNA Comm) depicted in Table 9 fully reproduces the inhibitory effect of SEVs produced by T-47D shCtl cells. Hetero-spheroids growth (A), Apoptosis (B) and Invasion capacity (C) of 2 TNBC cell line (MDA-MB-231/SUM-159PT) were performed as described in the experimental part of Example 2 with cell transfected with the miRNA Ctrl (miRNA Ctrl) or the combination of the 15 miRNAs (miRNA Comb) (n=6 for the MDA-MB-231, ****p<0.0001; n=3 for the SUM-159PT, ****p<0.0001, ***p<0.001.

### DETAILED DESCRIPTION OF THE INVENTION

In the context of the present invention, the inventors surprisingly found that several miRNAs contained in SEVs derived from cells overexpressing NFAT3 were involved in and able to reproduce the anticancer activity of the SEVs, a specific combination of 11 miRNAs being able to inhibit cancer cells motility, and 8 miRNAs being able to inhibit the growth of cancer cells/macrophages hetero-spheroids.

### Definitions

The terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”), in each case as used herein, when used to define products, methods and use, are open-ended and do not exclude additional, unrecited elements or method steps. Thus, a composition “comprising” one or more components may further comprise any additional unrecited component. “Consisting essentially of” shall mean excluding other components or steps of any essential significance. Thus, a composition “consisting essentially of” one or more components may further comprise only additional unrecited components without essential significance. “Consisting of” means excluding more than trace elements of other components or steps. Thus, a composition “consisting of” one or more components does not contain any additional unrecited components. In the context of the present disclosure, each time a product or method or use is indicated as “comprising” something, the embodiment in which said product or method or use consists essentially of or consists of the same something is also contemplated in the context of the invention.

“NFAT3”, “Nuclear factor of activated T-cells, cytoplasmic, calcineurin-dependent 4” or “NFATC4” as used herein refers to a protein encoded by the human gene with the official symbol NFATC4 in Entrez Gene database. (Gene ID: 4776) or variants thereof as defined below. The NFATC4 gene is also known as “nuclear factor of activated T-cells, cytoplasmic 4”, “T-cell transcription factor NFAT3”, “NFAT3”, “NF-AT3”, and “NF-ATC4”.

A “miRNA” or “microRNA” is a single-stranded molecule of about 18-25 nucleotides, preferably 21-23 in length, encoded by genes that are transcribed from DNA but not translated into protein (non-coding RNA); instead they are processed from primary transcripts known as pri-miRNA to short stem-loop structures called pre-miRNA and finally to functional mature miRNA. During maturation, each pre-miRNA gives rise to two distinct fragments with high complementarity, one originating from the 5′ arm the other originating from the 3′ arm of the pre-miRNA. Mature miRNA molecules are partially complementary to one or more messenger RNA (mRNA) molecules, and their main function is to downregulate gene expression.

There is an international nomenclature of miRNAs (see Ambros V et al, RNA 2003 9(3):277-279; Griffiths-Jones S. NAR 2004 32(Database Issue):D109-D111; Griffiths-Jones Set al. NAR 2006 34(Database Issue):D140-D144; Griffiths-Jones S et al. NAR 2008 36(Database Issue):D154-D158; and Kozomara A et al. NAR 2011 39(Database Issue):D152-D157), which is available from miRBase at http://www.mirbase.org/. Each miRNA is assigned a unique name with a predefined format, as follows:

For a mature miRNA: sss-miR-X-Y, wherein
“sss” is a three letters code indicating the species of the miRNA, “hsa” standing for human,
the upper case “R” in miR indicates that it is referred to a mature miRNA. However, some authors in the literature abusively use “mir” also for mature miRNA. In this case, it may be recognized that it is referred to a mature miRNA by the presence of “-Y”,
X is the unique arbitrary number assigned to the sequence of the miRNA in the particular species, which may be followed by a letter if several highly homologous miRNAs are known. For instance, “20a” and “20b” refer to highly homologous miRNAs.
Y indicates whether the mature miRNA, which has been obtained by cutting of the pre-miRNA, corresponds to the 5′ arm (Y is then “5p”) or 3′ arm (Y is then “3p”) of the pre-miRNA. In previous international nomenclature of miRNAs, “-Y” was not present. The two mature miRNAs obtained either from the 5′ or the 3′ arm of the pre-miRNA were then distinguished by the presence or absence of a “*” sign just after n. The presence of the “*” sign indicated that the sequence corresponded to the less often detected miRNA. Since such classification was subject to changes, a new nomenclature using the “3p” and “5p” code has been implemented.

For a pri-miRNA:sss-mir-X, wherein
sss is a three letters code indicating the species of the miRNA, “hsa” standing for human,
the lower case “r” in mir indicates that it is referred to a pri-miRNA and not to a mature miRNA, which is confirmed by the absence of “-Y”,
n is the unique arbitrary number assigned to the sequence of the miRNA in the particular species, which may be followed by a letter if several highly homologous miRNAs are known.

Each miRNA is also assigned an accession number for its sequence.

In the context of the present invention, miRNAs include both natural miRNAs and modified miRNAs that contain chemical modifications designed to enhance one or more of their properties.

In the present description, “cancer” refers to a malignant neoplasm characterized by deregulated or uncontrolled cell growth. In particular, a “cancer cell” refers to a cell with deregulated or uncontrolled cell growth.

The term “cancer” includes primary malignant tumours (also referred to as “primary cancer”, corresponding to those whose cells have not migrated to sites in the subject's body other than the site of the original tumor) and secondary malignant tumours (also referred to as “secondary cancer” or “metastatic cancer”, those arising from metastasis, the migration of tumour cells to secondary sites that are different from the site of the original tumour).

Among primary cancers, it may be distinguished between “non-aggressive” and “aggressive” primary cancers, the aggressivity of a primary cancer being defined as its propensity to metastasis, i.e. the higher the probability of metastasis, the higher is the primary cancer aggressiveness. Determining aggressiveness of the primary cancer of a subject is within the knowledge of cancer clinicians, and may notably be done based on the organ of origin of the primary cancer (some primary cancers are known to be more aggressive than others, such as pancreatic cancer), the histological staging of the primary cancer (the more advanced is the histological staging, the higher is the aggressiveness), (high) expression of one or more known aggressiveness markers, the absence or low expression of one or more known non-aggressiveness markers, and/or the clinical evolution of the cancer assessed by tumor size, presence of tumor cells in axillary lymph nodes, and/or metastatis. In the case of breast cancer, triple negative breast cancers (defined by the absence of expression or low expression by the tumor cells of estrogen receptor, progesterone receptor and HER2) are known to be more aggressive than luminal breast cancers.

In the present description, the term “treating” or “treatment” means an improvement of the patient's cancer, which may be observed at the clinical, histological, or biochemical level. In particular, any alleviation of a clinical, histological or biochemical symptom of the cancer is included in the terms “treating” and “treatment”.

In the context of primary cancer, “treating” or “treatment” thus notably relates to the fact to reduce cancer growth or spreading by metastasis. For instance, when referring to the treatment of primary cancer, “treatment” or “treating” may correspond to at least one of the following improvements:
a decrease or stabilization of tumor load compared to before administration of the pharmaceutical composition according to the invention,
a decrease or stabilization of a tumor biomarker (for instance PSA in prostate cancer) compared to before administration of the pharmaceutical composition according to the invention,
improved progression-free survival (PFS) or overall survival (OS), compared to what might be expected before administration of the pharmaceutical composition according to the invention,
absence of cancer spreading by metastasis,
delay of the onset or reduction of the intensity of cancer spreading by metastasis.

In the more precise context of metastatic cancer, “treating” or “treatment” notably relates to the fact to reduce metastatic cancer growth or further spreading by metastasis. For instance, “treatment” or “treating” of metastatic cancer may correspond to:
any of the improvements mentioned above in the general context of treating cancer,
a specific decrease in the metastasis load,
a decrease or stabilization of a metastasis biomarker.

Treatment may require administration of an agent and/or treatment more than once.

In the present description, the term “preventing” or “prevention” means the fact to preclude or delay the onset or reduce the intensity of clinical, histological or biochemical events associated with cancer. In the context of cancer, “preventing” or “prevention” thus notably relates to the fact to inhibit, at least partially, new cancer growth or spreading. When referring to metastatic cancer, “preventing” or “prevention” of metastatic cancer may correspond to the absence of cancer metastasis, or to the delay of the onset or reduction of the intensity of cancer metastasis compared to what might be expected before administration of the pharmaceutical composition according to the invention. Prevention may require administration of an agent and/ or treatment more than once.

A “therapeutically effective amount” corresponds to an amount necessary to impart therapeutic or preventive benefit to a subject, as defined above.

In the present description, the term “patient” or “subject” refers to mammals, e. g., humans, dogs, cows, horses, kangaroos, pigs, sheep, goats, cats, mice, rabbits, rats. In preferred embodiments of the present invention, the subject is a human subject.

miRNA Composition

The present invention first relates to a miRNA composition comprising the following 11 miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, and hsa-miR-7704.

The 11 miRNAs are present in the miRNA composition in isolated form, i.e. they are not included in another entity, such as a cell or a vesicle (including a secreted extracellular vesicle (SEV).

The miRNA composition thus preferably does not contain secreted extracellular vesicles or cells.

In particular, the miRNA composition preferably comprises the 11 above-mentioned miRNAs isolated in a buffer. Any buffer suitable for further loading of a delivery vector or for pharmaceutical use may be used. Suitable buffers include water, phosphate buffer saline (PBS), or Tris-EDTA. Water is preferred.

A preferred miRNA composition is thus a miRNA composition comprising in a buffer the following 11 isolated miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, and hsa-miR-7704.

For use in loading delivery vectors or for therapeutic use, the miRNA composition is preferably a liquid miRNA composition, preferably comprising the miRNAs in isolated form in a buffer. However, for storage purpose, the miRNA composition may be a solid or dry miRNA composition (for example a frozen or freeze-dried miRNA composition), comprising frozen or dried miRNAs, optionally in a frozen or dried buffer. This may permit storage at 4° C.

Said miRNA composition is able to inhibit cancer cells motility, and thus has antimetastatic properties. Moreover, 4 of the 11 miRNAs of the composition also have the ability to inhibit the growth of cancer cells/macrophages hetero-spheroids and thus have general anticancer properties: hsa-miR-16-5p, hsa-miR-196a-3p, hsa-miR-4488, and hsa-miR-7704.

miRNA Composition Comprising the 11 Minimal miRNAs Found to be Involved in Inhibition of Cancer Cells Motility

The miRbase accession number and sequence of each of the 11 miRNAs present in the miRNA composition according to the invention is presented in Table 1 below:

TABLE 1
miRbase accession number and sequence of each
of the 11 miRNAs present in the miRNA
composition according to the invention.

| | miRbase accession | |
|---|---|---|
| Name | number | Sequence |
| hsa-miR-3195 | MIMAT0015079 | CGCGCCGGGCCCGGGUU |
| | | (SEQ ID NO: 1) |
| hsa-miR-1246 | MIMAT0005898 | AAUGGAUUUUUGGAGCAGG |
| | | (SEQ ID NO:2) |
| hsa-miR-188-3p | MIMAT0004613 | CUCCCACAUGCAGGGUUUGCA |
| | | (SEQ ID NO: 3) |
| hsa-miR-16-5p | MIMAT0000069 | UAGCAGCACGUAAAUAUUGGCG |
| | | (SEQ ID NO: 4) |
| hsa-miR-196a-3p | MIMAT0004562 | CGGCAACAAGAAACUGCCUGAG |
| | | (SEQ ID NO: 5) |
| hsa-miR-32-5p | MIMAT0000090 | UAUUGCACAUUACUAAGUUGCA |
| | | (SEQ ID NO: 6) |
| hsa-miR-4532 | MIMAT0019071 | CCCCGGGGAGCCCGGCG |
| | | (SEQ ID NO: 7) |
| hsa-miR-4792 | MIMAT0019964 | CGGUGAGCGCUCGCUGGC |
| | | (SEQ ID NO: 8) |
| hsa-miR-4488 | MIMAT0019022 | AGGGGGCGGGCUCCGGCG |
| | | (SEQ ID NO: 9) |
| hsa-miR-326 | MIMAT0000756 | CCUCUGGGCCCUUCCUCCAG |
| | | (SEQ ID NO: 10) |
| hsa-miR-7704 | MIMAT0030019 | CGGGGUCGGCGGCGACGUG |
| | | (SEQ ID NO: 11) |

The 11 miRNAs of the miRNA composition according to the invention were present in specific ratios in the SEVs of cells expressing NFATC4, and the equilibrium between these 11 miRNAs might possibly be important for their inhibitory effect on the motility of cancer cells. Therefore, in a preferred embodiment, the 11 miRNAs are present in the composition in the relative ratios presented in Table 2 below.

TABLE 2
Preferred range of the relative ratio of each miRNA in the composition,
calculated with the ratio of hsa-miR-196a-3p set at 1.

| | Relative ratio in |
|---|---|
| miRNA | the miRNA composition |
| hsa-miR-3195 | 0.8 to 1.0, preferably 0.85 to 0.95 |
| hsa-miR-1246 | 0.5 to 0.7, preferably 0.55 to 0.65 |
| hsa-miR-188-3p | 0.5 to 0.7, preferably 0.55 to 0.65 |
| hsa-miR-16-5p | 0.5 to 0.7, preferably 0.55 to 0.65 |
| hsa-miR-196a-3p | 1.0 |
| hsa-miR-32-5p | 0.4 to 0.6, preferably 0.45 to 0.55 |
| hsa-miR-4532 | 0.5 to 0.7, preferably 0.55 to 0.65 |
| hsa-miR-4792 | 0.3 to 0.5, preferably 0.35 to 0.45 |
| hsa-miR-4488 | 0.5 to 0.7, preferably 0.55 to 0.65 |
| hsa-miR-326 | 0.4 to 0.6, preferably 0.45 to 0.55 |
| hsa-miR-7704 | 0.5 to 0.7, preferably 0.55 to 0.65 |

Even more preferably, the 11 miRNAs are present in the composition in the relative ratios presented in Table 3 below.

TABLE 3
Preferred relative ratio of each miRNA in the composition,
calculated with the ratio of hsa-miR-196a-3p set at 1.

| | Relative ratio in |
|---|---|
| miRNA | the miRNA composition |
| hsa-miR-3195 | 0.9 |
| hsa-miR-1246 | 0.6 |
| hsa-miR-188-3p | 0.6 |
| hsa-miR-16-5p | 0.6 |
| hsa-miR-196a-3p | 1.0 |
| hsa-miR-32-5p | 0.5 |
| hsa-miR-4532 | 0.6 |
| hsa-miR-4792 | 0.4 |
| hsa-miR-4488 | 0.6 |
| hsa-miR-326 | 0.5 |
| hsa-miR-7704 | 0.6 |

miRNA Composition Further Comprising at Least one of 4 Other miRNAs Found Involved in Inhibition of Hetero-Spheroid Growth

Moreover, the miRNA composition may preferably further comprise at least one of the 4 other mRNAs identified by the inventors as being able to inhibit the growth of cancer cells/macrophages hetero-spheroids, i.e. at least one miRNA selected from hsa-miR-195-5p, hsa-miR-324-3p, hsa-miR-4449 and hsa-miR-501-5p.

The miRNA composition according to the invention may thus further comprise:
one of hsa-miR-195-5p, hsa-miR-324-3p, hsa-miR-4449 and hsa-miR-501-5p,
any combination of two or three of hsa-miR-195-5p, hsa-miR-324-3p, hsa-miR-4449 and hsa-miR-501-5p:
   hsa-miR-195-5p and hsa-miR-324-3p,
   hsa-miR-195-5p and hsa-miR-4449,
   hsa-miR-195-5p and hsa-miR-501-5p,
   hsa-miR-324-3p and hsa-miR-4449,
   hsa-miR-324-3p and hsa-miR-501-5p, or
   hsa-miR-4449 and hsa-miR-501-5p,
any combination of two or three of hsa-miR-195-5p, hsa-miR-324-3p, hsa-miR-4449 and hsa-miR-501-5p;
   hsa-miR-195-5p, hsa-miR-324-3p, and hsa-miR-4449,
   hsa-miR-195-5p, hsa-miR-324-3p, and hsa-miR-501-5p,
   hsa-miR-195-5p, hsa-miR-4449, and hsa-miR-501-5p,
   hsa-miR-324-3p, hsa-miR-4449, and hsa-miR-501-5p, or
all of hsa-miR-195-5p, hsa-miR-324-3p, hsa-miR-4449 and hsa-miR-501-5p.

The miRbase accession number and sequence of each of the 4 additional miRNAs identified by the inventors as being able to inhibit the growth of cancer cells/macrophages hetero-spheroids, one or more of which may be present in the miRNA composition according to the invention is presented in Table 4 below:

TABLE 4
miRbase accession number and sequence
of each of the 11 miRNAs present in
the miRNA composition according to
the invention.

| | miRbase | |
|---|---|---|
| | accession | |
| Name | number | Sequence |
| hsa-miR- | MIMAT0000461 | UAGCAGCACAGAAAUAUUGGC |
| 195-5p | | (SEQ ID NO: 12) |
| hsa-miR- | MIMAT0000762 | CCCACUGCCCCAGGUGCUGCUGG |
| 324-3p⁽¹⁾ | | (SEQ ID NO: 13) or |
| | | ACUGCCCCAGGUGCUGCUGG |
| | | (SEQ ID NO:22) |
| hsa-miR- | MIMAT0018968 | CGUCCCGGGGCUGCGCGAGGCA |
| 4449 | | (SEQ ID NO: 14) |
| hsa-miR- | MIMAT0002872 | AAUCCUUUGUCCCUGGGUGAGA |
| 501-5p | | (SEQ ID NO: 15) |

⁽¹⁾For hsa-miR-324-3p, the sequence of the miRNA is not yet consensual and may be selected between the two indicated sequences depending on the consulted database (SEQ ID NO: 13 is now referenced on miRbase, while SEQ ID NO: 22 is still referenced on RNACentral, see rnacentral.org/rna/URS00004390F6/9606, and on TargetMiner, see www.isical.ac.in/~bioinfo_miu/final_html_targetminer/hsa-miR-324-3p.html). Any of the two sequences defined above may be thus used in the context of the invention, with a preference for SEQ ID NO:22, which has been used in the experimental section.

Optional Further miRNAs

The miRNA composition according to the invention may further comprise additional miRNAs. However, for practical reasons, the miRNA composition preferably comprises at most 100 distinct miRNAs, at most 75 distinct miRNAs, at most 50 distinct miRNAs, at most 40 distinct miRNAs, at most 30 distinct miRNAs, at most 25 distinct miRNAs, at most 20 distinct miRNAs, or even at most 15 distinct miRNAs.

Indeed, adding further miRNAs may alter the effect of the combination of 11 to 15 miRNAs according to the invention (the 11 minimal miRNAs, and 0 to 4 of the optional 4 additional miRNAs). Moreover, for in vivo administration, the miRNAs will preferably be loaded into a carrier (such as a liposome or an SEV), and the difficulty of the loading increases with the number of distinct miRNAs to be loaded.

The miRNAs comprised in the miRNA composition according to the invention therefore preferably consist essentially of or even consist of the combination of 11 to 15 miRNAs according to the invention (the 11 minimal miRNAs, and 0 to 4 of the optional 4 additional miRNAs).

A preferred miRNA composition comprises all of the following 15 miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, hsa-miR-7704, hsa-miR-195-5p, hsa-miR-324-3p, hsa-miR-4449 and hsa-miR-501-5p, preferably in the above-described ratios. The miRNAs comprised in this miRNA composition preferably consist of the 15 miRNAs, which are preferably in isolated form in a buffer, preferably selected from water, phosphate buffer saline (PBS), or Tris-EDTA (more preferably water).

Modified miRNAs

In order to improve their stability for in vivo uses, the miRNAs of the miRNA composition according to the invention may contain chemical modifications, including:
phosphorothioate-, rnethylphosphonate-, or boranophosphate-containing mRNA, which substitutes a sulfur, methyl, or borano group, respectively, for the α-oxygen of the phosphate,
introduction of a 2′-O-methyl or 2′-O-niethoxyethyl group to the ribose moiety or of 2′-fluoro into the 2′ oxygen of the ribose,
introduction of a 2′,4 methylene bridge in the ribose to form a bicyclic nucleotide, forming LNA,
terminal chemical modifications, including Cy3-, cholesterol-, biotin- and amino-modified RNA.
   Delivery Vectors Loaded With the miRNA Composition According to the Invention

The present invention also relates to a delivery vector loaded with the miRNA composition according to the invention.

In the present description, the term “delivery” corresponds to the fact to make the miRNA composition according to the invention available to cancer cells. A “delivery vector” (also referred to as a “delivery carrier”) is thus any means able to make the miRNA composition arrive to and penetrate into cancer cells.

Any type of delivery vector suitable for delivering miRNAs may be used, including:
viral delivery vectors, including adenoviral, adeno-associated, retroviral and lentiviral vectors, and
non-viral delivery vectors, including SEVs, liposomes, polymeric vectors/dendrimer-based vectors, inorganic material-based delivery systems, and 3D scaffold-based delivery systems.

In the context of the present invention, in view of the number of miRNAs to be delivered to cancer cells (at least 11, preferably 15), non-viral delivery vectors are preferred.

In addition, the use of liposomes or SEVs loaded with the miRNA composition according to the invention is preferred and is described in more details below.

Examples of viral vectors and other non-viral carriers suitable for the delivery of miRNAs are known in the art (see e.g. Forterre A, et al. Cancers (Basel). 2020 Jul. 9; 12(7):1852; O'Neill C P, Dwyer R M. Cells. 2020 Feb. 24; 9(2):521; Fu, Y., Chen, J. [symbol] Huang, Z. ExRNA 1, 24 (2019); Lee S W L, et al. J Control Release. 2019 Nov. 10; 313:80-95), and may be selected by those skilled in the art based on common general knowledge.

Liposomes Loaded With the miRNA Composition According to the Invention

The present invention also relates to liposomes containing the miRNA composition according to the invention.

In the present description, “liposomes”, “lipid nanoparticles” or “LNP”, and “lipid-based nanocarriers” are used as synonyms and relate to lipid vesicles consisting of one or more phospholipid bilayers encapsulating an aqueous solution. The advantages of liposomes as delivery agents include biocompatibility, flexibility, low immunogenicity, and versatility of administration routes.

Their efficiency depends on physicochemical properties such as particle size, surface charge and lipid composition.

In particular, liposomes can be differentiated based on vesicle charge. Cationic lipids incorporated into liposomes facilitate strong binding to the anionic phosphate backbone of miRNAs and can provide more efficient delivery by binding to anionic molecules on the target cell surface. However, due to this high reactivity with anionic molecules, there have been reports of immunogenicity. Neutral liposomes, as the name suggests, have no charge and are believed to be less immunogenic. In addition, they may have improved tumor accumulation as compared to cationic liposomes due to reduced RES clearance and prolonged circulation. Ionizable liposomes are cationic at low pH, and neutral or anionic at neutral or higher pH, and can selectively enhance cellular uptake.

Liposomes used for delivering the miRNA composition according to the invention preferably have a diameter lower or equal to about 250 nm, in particular between about 30 and about 250 nm. Such nanometer range size may be obtained using sonication.

Lipids used in liposomes for in vivo delivery include:
cationic lipids, such as didodecyldimethylammonium bromide (DDAB), 1,2-distearoyl-3-dimethylammonium-propane (DSDAP), 1,2-dioleoyl-3-trimethylammonium-propane (DOTAP), 1,2-distearoyl-sn-glycero -phosphatidylcholine (DSPC) and 1,2-distearoyl-sn-glycero-3-phosphoryl ethanolamine (DSPE),
neutral lipids (also referred to as “helper lipids”, such as cholesterol (Chol), dioleoylphosphatidyl ethanolamine (DOPE), phosphatidylcholine (PC), glycerol-monooleate (GMO), and 1,2-dioleoyl-sn-glycerophosphatidylcholine (DOPC), and
ionizable lipids, such as DLin-KC2-DMA (2,2-dilin-oleyl-4-(2-dimethylaminoethyl) -[1,3]-dioxolane) with a pKa of 6.7, and DLin-MC3-DMA (1,2-dilinoleyloxy-N,N -dimethyl-3-aminopropane) with a pKa of 6.4.

(Cationic lipids or ionizable lipids) and neutral lipids are preferably used in a (cationic lipids or ionizable lipids)/neutral lipids ratio comprised between 0.5/1 and 4/1, preferably between 2/3 and 2/1.

Liposomes may further be modified with other molecules, including hyaluronic acid (HA) and polyethylene glycol (PEG), to improve characteristics such as tumor targeting and stability. PEG may notably be attached to a cationic or neutral lipid.

miRNA-loaded liposomes may notably be made with a mixture of 1) cationic lipids or ionizable lipids, 2) neutral lipids and optionally 3) PEG.

Methods for preparing liposomes are known in the art, and may be selected by those skilled in the art based on common general knowledge.

A method that may be used for preparing miRNA-liposomes comprises (Lujan H, et al. International Journal of Nanomedicine. 2019 ;14:5159-5173):
mixing a cationic or ionizable lipid, a neutral lipid and optionally PEG (which may be attached to a cationic, ionizable or neutral lipid) in suitable concentrations and ratios in 100% ethanol;
adding the miRNA(s) to be loaded and incubating at a temperature of 10° C. to 40° C., preferably of 15° C. to 30° C., more preferably of 18 to 25° C., during 15 minutes to 2 hours, preferably 20 minutes to 1 hour, in particular about 30 minutes;
rapidly injecting the obtained solution into stirred PBS and vigorously stirring (such as at 1000 to 2000 rpm, for instance 1400 rpm) the obtained solution during 3 to 10 minutes, such as about 5 minutes,
dialyzing the resultant miRNA-loaded liposome suspension solution in PBS, and
optionally, sonicating the resultant miRNA-loaded liposome suspension, preferably at 30-40 kHz (such as 37 kHz) for 5 to 15 minutes (such as 10 minutes), in order to produce miRNA-loaded liposomes with a size in the nanometer range.
   SEVs Loaded With the miRNA Composition According to the Invention

The cargo of SEV is potentially particularly interesting for targeting therapies to tumors, as SEV are secreted in the extracellular compartment, where their content is protected from degradation because of their lipid membrane, and SEV excreted from one cell are known to be able to fuse with surrounding cells, and thus have the potential to initiate signaling responses (Hendrix, A. et al. Cancer Res. 70, 9533-9537 (2010)).

Therefore, the present invention also relates to secreted extracellular vesicles (SEVs) loaded with the miRNA composition according to the invention.

By “secreted extracellular vesicles” or “SEV”, it is meant membranous vesicles released by cells in their microenvironment from their plasma membrane. In the context of the present invention, SEV typically have a diameter lower or equal to about 500 nm, in particular between about 30 and about 500 nm, or between about 40 and about 500 nm, or between about 50 and about 500 nm. SEV are surrounded by a phospholipid membrane, which preferably contains relatively high levels of cholesterol, sphingomyelin, and ceramide and preferably also contains detergent-resistant membrane domains (lipid rafts).

The membrane proteins of SEV have the same orientation as the cell. SEV are generally characterized by the presence of Actin β, proteins involved in membrane transport and fusion (such as Rab, GTPases, annexins, and flotillin), components of the endosomal sorting complex required for transport (ESCRT) complex (such as Alix), tumor susceptibility gene 101 (TSG101), heat shock proteins (HSPs, such as HSPA8, HSP9OAA1, HSC70 and HSC90), integrins (such as CD62L, CD62E or CD62P), and tetraspanins (in particular CD63, CD81, CD82, CD53, CD9, and/or CD37).

SEV may also naturally contain RNAs, such as mRNAs and miRNAs.

The SEVs according to the invention have been loaded with the miRNA composition according to the invention. Due to this loading, they contain higher concentrations in the miRNAs of the miRNAs composition according to the invention than the SEVs produced by cells expressing NFAT3 disclosed in WO2017167788A1 and de Camargo, L. C. B et al. Sci Rep 10, 8964 (2020).

### Method for Preparing the Loaded SEVs According to the Invention

The present invention also relates to a method for preparing the loaded SEVs according to the invention, comprising:
a) preparing SEVs from healthy cells,
b) loading the SEVs with a miRNA composition according to the invention.

### Step a): Preparing SEVs From Healthy Cells

In step a), SEVs are prepared from healthy cells, preferably healthy human cells when the treated subject is a human subject.

Appropriate human healthy cells include HEK293T and WI38 cells. SEVs may be prepared from cells secreting them by various methods, the most common and most preferred of which is differential centrifugation. Methods that may be used for preparing SEVs according to the present invention from healthy cells in culture include, as disclosed in Yakimchuk, K. Mater. Methods, 2015, vol. 5: 1450:

### Differential Centrifugation:

As explained above, this method is one of the most common techniques and the preferred method in the present invention.

The method consists of several steps, preferably performed at about 4° C., including at least the following three steps 1) to 3):
1) a low speed centrifugation to remove cells and cellular debris,
30 2) a higher speed spin to eliminate larger vesicles (size over 100 nm), and finally
3) high speed centrifugation to pellet SEV.

In step 1), cells and cellular debris are removed using centrifugal accelerations of about 1300 to 3500 RPM (rounds per minute) for 5-30 minutes. Optionally, step 1) may include two low speed centrifugations, the first at very low speed (for instance about 1350 RPM) and the second at highest speed (for instance 3500 RPM).

In particular, spinning at 1350 RPM for 10 minutes at about 4° C. followed by spinning at 3500 RPM for 20 minutes at about 4° C. may be used in step 1).

In step 2), larger vesicles (size over 100 nm) are eliminated using centrifugal accelerations of about 10 000 RPM for 15-45 minutes. In particular, spinning at about 10,000 RPM for 30 minutes at about 4° C. may be used in step 2).

In step 3), which is preferably performed at least twice, SEV are pelleted using centrifugal accelerations of about 40 000 RPM (for 60-120 minutes. In particular, spinning at 40,000 RPM for 90 minutes at about 4° C. may be used in step 3).

A particularly preferred protocol is as described below:
a) Spin cultured cells at 1350 RPM for 10 minutes at 4° C. and collect supernatant;
b) Spin collected supernatant at 3500 RPM for 20 minutes at 4° C. and collect supernatant;
c) Spin collected supernatant at 10,000 RPM for 30 minutes at 4° C. and collect supernatant;
d) Ultracentrifuge collected supernatant at 40,000 RPM for 90 minutes at 4° C. and collect the pellet;
e) Resuspend the pellet in cold PBS; and
f) Ultracentrifuge at 40,000 RPM for 90 minutes at 4° C. and collect the pellet.

### Density Gradient Centrifugation:

This approach combines ultracentrifugation with sucrose density gradient.

More specifically, density gradient centrifugation is used to separate SEV from non-vesicular particles, such as proteins and protein/RNA aggregates. Thus, this method separates vesicles from the particles of different densities. The adequate centrifugation time is very important, otherwise contaminating particles may be still found in SEV fractions if they possess similar densities. Recent studies suggest application of the SEV pellet from ultracentrifugation to the sucrose gradient before performing centrifugation.

### Size Exclusion Chromatography:

Size-exclusion chromatography is used to separate macromolecules on the base of size, not molecular weight. The technique applies a column packed with porous polymeric beads containing multiple pores and tunnels. The molecules pass through the beads depending on their diameter. It takes longer time for molecules with small radii to migrate through pores of the column, while macromolecules elute earlier from the column. Size-exclusion chromatography allows precise separation of large and small molecules. Moreover, different eluting solutions can be applied to this method.

### Filtration:

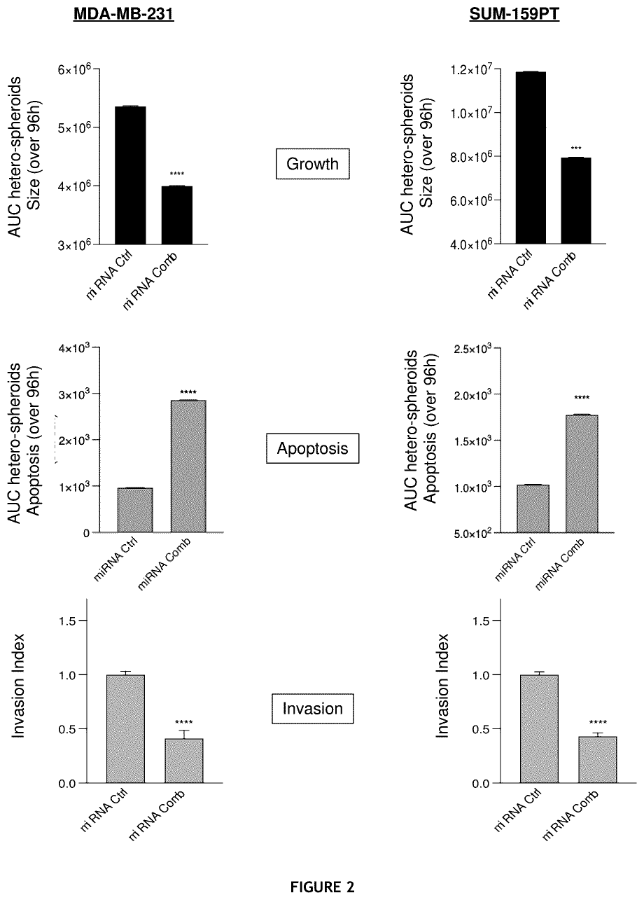

Ultrafiltration membranes can also be used for isolation of SEV. Depending on the size of microvesicles, this method allows the separation of SEV from proteins and other macromolecules. SEV may also be isolated by trapping them via a porous structure (FIG. 2). Most common filtration membranes have pore sizes of 0.8 μm, 0.45 μm or 0.22 μm and can be used to collect SEV larger than 800 nm, 400 nm or 200 nm. In particular, a micropillar porous silicon ciliated structure was designed to isolate 40-100 nm SEV. During the initial step, the larger vesicles are removed. In the following step, the SEV population is concentrated on the filtration membrane. The isolation step is relatively short, but the method requires pre-incubation of the silicon structure with PBS buffer. In the following step, the SEV population is concentrated on the filtration membrane.

### Polymer-Based Precipitation:

Polymer-based precipitation technique usually includes mixing the biological fluid with polymer-containing precipitation solution, incubation at 4° C. and centrifugation at low speed. One of the most common polymers used for polymer-based precipitation is polyethylene glycol (PEG). The precipitation with this polymer has a number of advantages, including mild effects on isolated SEV and usage of neutral pH. Several commercial kits applying PEG for isolation of SEV were generated. The most commonly used kit is ExoQuick™ (System Biosciences, Mountain View, CA, USA). This kit is easy and fast to perform and there is no need for additional equipment. Recent studies demonstrated that the highest yield of SEV was obtained using ultracentrifugation with ExoQuick™ method.

### Immunological Separation:

Several techniques of immunological separation of SEV have been developed, based on surface SEV receptors or SEV intracellular proteins. These methods are however generally applied mainly for detection, analysis and quantification of SEV proteins.

### Isolation by Sieving:

This technique isolates SEV by sieving them from biological liquids via a membrane and performing filtration by pressure or electrophoresis.

Step b): Loading the SEVs With a miRNA Composition According to the Invention

Loading of the SEVs with a miRNA composition according to the invention may be performed using any suitable method known in the art.

These include mixing miRNA(s) and SEVS and using electroporation or CaCl₂ transfection for introducing the miRNA(s) into the SEVs (see Zhang D, et al. Am J Physiol Lung Cell Mol Physiol. 2017 Jan. 1; 312(1):L110-L121. doi: 10.1152/ajplung.00423.2016. Epub 2016 Nov. 23. PMID: 27881406; PMCID: PMC5283929).

However, improved methods for loading SEVs with miRNAs have been developed, two of which are described in more details below.

Therefore, the loading of the SEVs preferably involves:
pH gradient modification of the SEVs, or
creating a turbulent flow in the liquid medium containing:
   the SEVs, wherein the Kolmogorov length of the flow is less than or equal to 100 μm, or
   the cells producing the SEVs, wherein the Kolmogorov length of the flow is less than or equal to 50 μm.
      First Preferred Method for Loading the SEVs With a miRNA Composition According to the Invention, Based on pH Gradient Modification of the SEVs

A first preferred method permits enhanced loading of miRNAs in SEVs based on pH gradient modification of the SEVs.

Therefore, in a preferred embodiment, step a) and step b) are performed sequentially and step b) comprises the following consecutive substeps i) to iv), as disclosed in Jeyaram A. et al. Mol Ther. 2020 Mar. 4; 28(3):975-985:
i) dehydrating SEVs in an alcohol, preferably ethanol,
ii) rehydrating SEVs in an acidic buffer of pH 2 to 3,
iii) dialyzing SEVs with a neutral buffer of pH 6.5 to 7.5, and
iv) incubating SEVs with the miRNA composition according to the invention, preferably at a temperature of 10 to 40° C. and preferably during 30 minutes to 6 hours.

In substep i), SEVs prepared in step a) are dehydrated in ethanol. In particular, SEVs may be dehydrated in 60-80% ethanol, preferably in 65-75% ethanol, such as 70% ethanol.

In substep ii), dehydrated SEVs are rehydrated in an acidic buffer of pH 2 to 3. For instance, citrate buffer is an appropriate acidic buffer of pH 2 to 3. The pH of the acidic buffer may in particular be comprised between 2.1 to 2.9, between 2.2 to 2.8, preferably between 2.3 to 2.7, between 2.4 to 2.6, such as pH 2.5. In particular, citrate buffer of pH 2.5 may preferably be used in substep ii).

In substep iii), rehydrated SEVs are dialyzed with a neutral buffer of pH 6.5 to 7.5. For instance, HEPES buffer is an appropriate neutral buffer of pH 6.5 to 7.5 include HEPES buffer. The pH of the neutral buffer may in particular be comprised between 6.6 to 7.4, between 6.7 to 7.3, preferably between 6.8 to 7.2, between 6.9 to 7.1, such as pH 7.0. In particular, HEPES buffer of pH 7.0 may preferably be used in substep iii).

Finally, in substep iv), dialyzed SEVs are incubated with the miRNA composition according to the invention. The incubation is preferably made at a temperature of 10 to 40° C., preferably of 15° C. to 37° C., of 18° C. to 30° C., or of 20° C. to 25° C. Alternatively or preferably in combination, the incubation is preferably maintained during 30 minutes to 6 hours, more preferably during 30 minutes to 4 hours, or during 1 hour to 2 hours.

Second Preferred Method for Loading the SEVs With a miRNA Composition According to the Invention, Based on Creation of a Turbulent Flow in the Liquid Medium

A second preferred method permits enhanced loading of miRNAs in SEVs using a turbulent flow of the liquid medium containing the SEVs or the cells producing the SEVs.

In a first alternative of this second method, step a) and step b) are performed sequentially and step b) comprises the following consecutive substeps i) to iii) as disclosed in WO2020/136361:
i) providing SEVs in a liquid medium comprising the miRNA composition, and
ii) agitating the liquid medium under agitation conditions causing a turbulent flow of the liquid medium, the Kolmogorov length of the flow being less than or equal to 100 μm, preferably less than or equal to 80 μm, less than or equal to 70 μm, or less than or equal to 60 μm, said flow making it possible to load the miRNA composition into the SEVs, and
iii) collecting the loaded SEVs.

More details about this method, as well as devices for implementing the method are disclosed in WO2020/136361, the content of which is herein incorporated by reference.

In a second alternative, step a) and step b) are performed simultaneously and comprise:
i) cultivating healthy cells in a liquid medium comprising the miRNA composition according to the invention under agitation conditions causing a turbulent flow of the liquid medium, the Kolmogorov length of the flow being less than or equal to 50 μm, preferably less than or equal to 40 μm, said flow making it possible to simultaneously load the miRNA composition and produce the SEVs, and
ii) collecting the loaded SEVs.

More details about this method, as well as devices for implementing the method are disclosed in WO2020/136362, the content of which is herein incorporated by reference.

### Pharmaceutical Composition

The present invention also relates to a pharmaceutical composition comprising the miRNA composition, the liposome, or the SEV according to the invention or prepared using the method according to according to the invention or mixtures thereof.

In addition to the miRNA composition, the liposome, or the SEV according to the invention or prepared using the method according to according to the invention or mixtures thereof, the pharmaceutical composition according to the invention preferably also contains a pharmaceutically acceptable carrier.

Such pharmaceutically acceptable carrier will be selected by those skilled in the art based on their common general knowledge depending on the specific therapeutic agent of the composition (miRNAs, liposomes, SEVs or mixtures thereof) and the selected administration route.

### Therapeutic Uses

Based on the anticancer effect of the miRNA composition according to the invention, the present invention also relates to a pharmaceutical composition according to the invention, for use as a medicament.

### Treatment of Cancer

The inventors have shown that a composition comprising the minimal 11 miRNAs is efficient in inhibiting cancer cells motility and that 4 of the 11 mRNAs were also able independently to inhibit the growth of cancer cells/macrophages hetero-spheroids. The pharmaceutical composition according to the invention is thus expected to be efficient in the treatment of cancer.

The present invention thus also relates to a pharmaceutical composition according to the invention, for use in the treatment of cancer.

The present invention also relates to the use of a pharmaceutical composition according to the invention in the manufacture of a medicament for the treatment of cancer.

The present invention also relates to the use of a pharmaceutical composition according to the invention for the treatment of cancer.

The present invention also relates to a method of treatment of cancer in a subject in need thereof, comprising administering to the subject a therapeutically efficient amount of a pharmaceutical composition according to the invention.

### Prevention or Treatment of Cancer Metastasis

As the minimal 11 miRNAs comprised in the pharmaceutical composition according to the invention are efficient in inhibiting cancer cells motility, the pharmaceutical composition according to the invention is also expected to be efficient in the prevention and treatment of cancer metastasis.

The present invention also relates to a pharmaceutical composition according to the invention, for use in the prevention or treatment of cancer metastasis.

The present invention also relates to the use of a pharmaceutical composition according to the invention in the manufacture of a medicament for the prevention or treatment of cancer metastasis.

The present invention also relates to the use of a pharmaceutical composition according to the invention for the prevention or treatment of cancer metastasis.

The present invention also relates to a method of prevention or treatment of cancer metastasis in a subject in need thereof, comprising administering to the subject a therapeutically efficient amount of a pharmaceutical composition according to the invention.

### Type of Cancer

The type of cancers that may be treated or of metastatic cancer that may be treated or prevented using a pharmaceutical composition according to the invention is not particularly limited.

Such cancer may notably be selected from the group of solid cancers. Solid cancers notably include carcinomas (cancers that begin in the lining layer (epithelial cells) of organs, glands, or body structures, also known as “epithelial cancers”), sarcomas (cancers that start in connective tissue, such as cartilage, fat, muscle, tendon, or bone), and brain cancers (cancers that start in brain cells, such as glioma, glioblastoma, and astrocytoma).

A cancer is further named after the part of the body where it originated. When cancer spreads, it keeps this same name. In the context of the invention, the cancer may in particular be selected from the group of carcinomas, including but not limited to breast carcinoma, melanoma, ovarian carcinoma, digestive carcinomas (also referred as gastrointestinal carcinomas, including colorectal carcinoma, oesophageal carcinoma, gastric carcinoma, pancreatic carcinoma, hepatocellular carcinoma, cholangiocellular carcinoma and teratocarcinoma), lung carcinoma, prostate carcinoma, and throat carcinoma, particularly of human subject. In the context of the invention, the solid cancer may also be selected from the group of brain tumors, including but not limited to glioblastoma, particularly of human subject. In preferred embodiments, the solid cancer may in particular be selected from breast carcinoma, melanoma, pancreatic carcinoma, colorectal carcinoma, glioblastoma and lung carcinoma; more preferably said cancer is selected from breast carcinoma, melanoma, pancreatic carcinoma, and glioblastoma, most preferably said solid cancer is breast carcinoma, in particular metastatic breast carcinoma, particularly of human subject.

The cancer may however also be selected from the group of hematopoietic cancers, and in particular from the group consisting of leukaemias, lymphomas, and myelomas, particularly of human patient.

Among primary cancers, the pharmaceutical composition according to the invention may preferably be used in the case of aggressive cancer. In particular, breast cancer (in particular triple negative breast cancer), melanoma, pancreatic cancer, glioblastoma, colorectal cancer, and lung cancer may be aggressive, and may thus be preferably treated in the context of the invention.

### Administration Route

The pharmaceutical composition according to the invention may be administered by any suitable administration route, including intravenous, intratumoral, topical, intranasal, rectal, oral, transdermal, subcutaneous, and sublingual routes.

In a preferred embodiment, the pharmaceutical composition according to the invention is administered by intravenous or intratumoral route.

### Doses

The administered dose may vary depending on the therapeutic product used (miRNA composition alone, specific type of delivery vector), subject age, body surface area or body weight, or on the administration route and associated bioavailability. Such dose adaptation is well known to those skilled in the art.

The following examples merely intend to illustrate the present invention.

### EXAMPLES

### Example 1: Analysis of miRNAs Specifically Overexpressed in the Antitumoral SEVs of Cells Expressing NFAT3, and of Their Inhibitory Effects on the Motility Of Cancer Cells, and on the Growth of Cancer Cells/Macrophages Hetero-Spheroids

### Materials and Methods

Cell culture

Highly metastatic triple negative human breast carcinoma cell line MDA-MB-231 were used, as well as a stable shRNA control line (T-47D-shCtl) produced in the laboratory. All the cell lines were cultivated in complete medium. The MDA-MB-231 were maintained in low glucose (1 g /L D-glucose) DMEM (Dulbecco's modified Eagle's medium), supplemented with 10% fetal calf serum, 2 mM glutamine, penicillin (100 U/mL)—streptomycin (100 ug/mL) and 100 μg/μL normocin (anti-mycoplasma), in a humid atmosphere incubator at 37° C. and 5% CO2. The T-47D-shCtl cell line was cultured in RPMI Medium 1640, 10% FCS, 2 mM of glutamine, penicillin (100 U/mL)—streptomycin (100 ug/mL) and 100 μg/μL of normocin and 1.5 ug/mL of puromycine. Puromycine and normocin were omitted when SEV were produced). Tests for the absence of contamination by mycoplasmas are carried out regularly.

### SEV Production

### Preparation of SEV Depleted Medium

For media containing animal driven components (e.g. serum), SEV depletion of the medium should be conducted.
1. Prepare medium with up to 20% serum (higher serum concentration is not recommended).
2. Precool the ultracentrifuge and rotor to 4° c.

### For SW32Ti Rotor:

i. Use pollyallomer 38.5 ml tubes (326823). Fill each with 38 ml medium and balance by weight (aim for 0.01-0.02 gr differences).
ii. Spin overnight (for standardization—18 hours) at 4° c at 30,000 RPM.

### For Type 45Ti:

i. Use 65 ml reusable tubes labeled for depletion. Fill each with 65 ml medium and balance by weight (aim for 0.01-0.02 gr differences).
ii. Spin overnight (for standardization—15 hours) at 4° c at 38,000 RPM.
3. Take supernatant carefully, do not disturb the pellet.
4. Filter 0.22 μm and save at 4° c. Use within 1 week.

### Medium Change

Grow cells as optimized for SEV production for the cell type, change to SEV depleted in the time point specified.
1. Prepare medium from SEV depleted medium, dilute if necessary.
2. Vacuum medium.
3. For 150 mm dish, add 7 ml PBS.
4. Vacuum well all PBS or medium left.
5. Add SEV depleted medium.

### SEV Production

Grow cells as optimized for SEV production for the cell type, change to SEV depleted medium if necessary and conduct the SEV isolation at the time point indicated (typically 24-48 hours after medium change).

Note: Cells should be in good condition—cell death and apoptotic bodies could lead to contamination of SEV pellet.

If SEV are to be used for functional assays, conduct in sterile conditions.

Precool ultracentrifuge with rotor Type 45Ti (this rotor takes a long time to cool, it is better to leave it on the fridge the night before starting the production).
1. Pass medium from dishes to 50 ml tubes.
2. Spin 1350 RPM for 10 minutes at 4° c.

In parallel: For a representative number of dishes (typically—3 for a 30 dishes production):
a. Wash with PBS and vacuum
b. Add trypsin, incubate 37° c until detachment
c. Add medium with 10% serum, preferably SEV depleted
d. Count cells from each dish separately and keep record of total cells used for production.
e. Add cold PBS
f. Centrifuge 1350 RPM for 7 minutes at 4° c.
g. Vacuum supernatant
h. Freeze −80° c for protein/RNA.
3. Pass supernatant carefully to new 50 ml tubes. Discard tubes with pellet.
4. Spin 3500 RPM in culture room) for 20 minutes at 4° c.
5. Pass supernatant carefully to new 50 ml tubes. Discard tubes with pellet.
6. In centrifuge 5810R rotor F-34-6-38: Spin 10,000 RPM for 30 minutes at 4° c.
7. Pass supernatant carefully to sterile ultracentrifuge tubes for rotor Type 45 Ti (tubes 355622). Look carefully that tubes are not cracked. Fill tubes with 65 ml and balance by weight (aim for 0.01-0.02 gr differences). Mark the tube in the side close to rotor in order to mark pellet location.

At this step the medium can be kept in 4° c for a few days (max 3-4) before ultracentrifugation.
8. Ultracentrifuge 40,000 RPM for 90 minutes at 4° c.
9. Carefully, as far as possible from pellet location—discard supernatant, leaving the minimal volume possible (if you can see the pellet, aspirate completely, if not, leave max 2 mL medium).
10. Using a p1000, resuspend the pellet in 2 mL cold PBS (or the remaining supernatant) and pass to another tube.
11. Add 2 ml cold PBS and wash with p1000, taking care to wash carefully the marked pellet location. Pass to the same tube.
12. Repeat wash with 2 ml PBS and pass to the same tube.
13. Repeat for all tubes, passing all to the same tube.
14. Fill tube to 65 ml with cold PBS and balance weight.
15. Ultracentrifuge 40,000 RPM for 90 minutes at 4° c.
16. Carefully, as far as possible from pellet location - discard supernatant, leaving the minimal volume possible.
17. Resuspend in remaining supernatant or add cold PBS in minimal amount to resuspend (this will depend on amount of cells used and cell type). Take care to wash carefully the marked pellet location. Pass to a sterilized siliconized tube (sigma T3281).
18. Wash tube, especially pellet location, with minimal amount of cold PBS. Pass to same siliconized tube.
19. Measure final volume and keep record.
20. Aliquot into sterile siliconized tubes and keep at −80° c.

### Important Observations:

For MDA-MB 231 and SUM 159 PT cells: seed 1×10E6 cells per 150 mm diameter dish; 3 days later change the medium to SEV depleted; 2 days later start the production;

For T47-D cells: seed 6×10E6 cells per 150 mm diameter dish; 5 days later change the medium to SEV depleted; 2 days later start the production;

For MCF7 cells: seed 7×10E6 cells per 150 mm diameter dish; 5 days later change the medium to SEV depleted; 2 days later start the production;

When using 150 mm diameter dish, keep cells in 25 mL medium;

For all of the cell types listed above, a visible pellet is obtained even after the 1st ultra-centrifugation, so it is important to eliminate all supernatant both after the 1st ultra and after the PBS wash to avoid contamination with proteins that could still be in suspension in the supernatant;

SEV depleted RPMI and DMEM are prepared with 20% serum; 1% P/S (and l-glu, in the case of RPMI). The medium is filtered after the ON ultracentrifugation and can be kept in the fridge up to 1 month. When diluting 20% SEV depleted medium before changing cell medium for production, a new bottle of fresh medium should be used. The diluted medium should be re-filtered upon dilution before changing the cell medium for the production.

### Cell Transfection

The MDA-MB-231 cells were seeded in 96-well plates (18,750 cells/well) and cultured for 24 hours in 200 μL of complete medium without normocin. 24 hours later, the cells were transfected with Dharmafect-1 as lipofectant. For the loss of function experiment (antagomir), cells were transfected with either an antagomir control or with each of the antagomir targeting the 21 miRNAs depicted in Table 5 at 25 nM; for the gain of function experiments cells were transfected with either a miRNA control or with each of the 21 miRNAs depicted in Table 5 at 25 nM. For each miRNA, the corresponding antagomir was a single-stranded RNA 100% complementary to the sequence of the miRNA of interest (these sequences are disclosed in Tables 1, 4 and 5, for hsa-miR-324-3p, the sequence SEQ ID NO:22 was used as sequence of the miRNA) and chemically modified with: 2 phosphorothioates at the 5′ end, 4 phosphorothioates at the 3′ end, a cholesterol group at the 3 ‘end and a full-length 2’-O-methyl nucleotide modification.

### Scratch Wound Assay

24 h after transfection MDA-MB-231 cell were washed with 200 μL of MEM without SVF. Then, the cells were pretreated for 24 hours with the SEV at a concentration of 3,10⁸ particles/ml for the conditions with treatment, in a final volume of 10 μL of MEM 2.5% FCS. For the control conditions, only 2.5% SVF MEM was added. The next day, the wound was made using the IncuCyte® WoundMaker, a 96-pick mechanical device designed to create homogeneous scratch wounds of 800 microns wide. The wells were washed once with 100 μL of MEM without SVF. Then 100 μL of 2.5% FCS MEM was added in each well. The plates were then placed in the IncuCyte. Images were acquired every 30 min for 24 hours. Image analysis was done on ImageJ software which allows us to calculate the area of the wound for each recorded time points.

### Hetero-Spheroid Growth Assay and Apoptosis Detection

For hetero-spheroids growth and apoptosis detection 5×10³ transfected MDA-MB-231+2.5×10³ RAW264.7 cells were seeded in a 96-well ultra-low attachment plates (Corning, #7007) in 100 μL in their corresponding medium containing 3% FBS and 3% Matrigel and let for 3 days in the incubator to allow the hetero-spheroids to form. Then medium containing the fluorescent marker of activated caspase 3/7 (Green INCUCYTE caspace-3/7, Essen Bioscience) was added for 6h to the wells in order to obtain a homogeneous labeling of the cells. Six hours later, medium containing SEVs (3,10⁸ particles/nil) or not was added to the wells. Green fluorescent images were acquired on the INCUCYTE device (Essen Bioscience) every 2 hours for 4 days. Image analysis was done on ImageJ software which allows us to calculate the area of the hetero-spheroid and the green fluorescence for each recorded time points. AUC of hetero-spheroids growth and apoptosis were calculated with the GraphPad Prism software for each tested condition for a total time of 4 days.

### Immunofluorescence.

At the end of mice in vivo experiments (around 60 days after cell implantation in the fat pad), tumors dissection and frozen tumor tissues sections were performed. Briefly, slides were fixed in 4% paraformaldehyde, saturated for non-specific binding by incubation in blocking buffer (PBS, 1% BSA, 10% Donkey serum, 0.1% TX-100) for 1 h at room temperature. Then, the slides were incubated with specific antibodies to mouse macrophages anti-F4/80 antibody (1:100, no. AB1140040, Abcam) in blocking buffer overnight at 4° C. The following day, the slides were washed and incubated with Alexa Fluor 594 Donkey anti-Rat IgG (H+L) Secondary Antibody (diluted 1:200, Abcam, # ab6640) in blocking buffer for 1 hour at room temperature. The slides were then washed, and non-specific binding was blocked in blocking buffer (PBS, 1% BSA, 10% mouse serum, 0.1% TX-100) for 1 h. The slides were then incubated for 1 h at room temperature with Anti-Pan human cytokeratins Alexa Fluor 488 antibody. After washes, slides were mounted in Fluoromount G mounting media with diluted DAPI (1:1000; no. AB259536, Sigma-Aldrich).

### Results

Identification of miRNAS Overexpressed in SEVs of Cells Expressing NFAT3 Compared to SEVs of Cells not Expressing NFAT3

Based on RNA-seq experiment, 21 specific miRNAs up-regulated in the inhibitory EVs originated from T47D shCtl (low invasive breast cancer cells naturally expressing NFAT3) compared to those produced in T47D shNFAT3 (in which the expression of NFAT3 is knocked down, i.e. cells that do not express NFAT3) were identified and are presented in Table 5 below:

TABLE 5
21 miRNAs up-regulated in EVs produced in
T-47D shCtl compared to those present in
EVs produced in T-47D shNFAT3 with fold
increase reported by the RNA seq

| miRNAs up- | | |
|---|---|---|
| regulated | Fold | |
| in T47D | increase | |
| shCtl SEVs | compared | |
| compared | to | |
| to miRNA | miRNA in | |
| in T47D | T-47D | |
| shNFAT3 | shNFAT3 | |
| SEVs | SEVs | miRNA sequence |
| hsa-miR-1246 | 2.13 | AAUGGAUUUUUGGAGCAGG |
| | | (SEQ ID NO: 2) |
| hsa-miR-1277-3p | 2.155 | UACGUAGAUAUAUAUGUAUUUU |
| | | (SEQ ID NO: 16) |
| hsa-miR-16-5p | 2.045 | UAGCAGCACGUAAAUAUUGGCG |
| | | (SEQ ID NO: 4) |
| hsa-miR-188-3p | 2.13 | CUCCCACAUGCAGGGUUUGCA |
| | | (SEQ ID NO: 3) |
| hsa-miR-188-5p | 1.735 | CAUCCCUUGCAUGGUGGAGGG |
| | | (SEQ ID NO: 17) |
| hsa-miR-195-5p | 3.735 | UAGCAGCACAGAAAUAUUGGC |
| | | (SEQ ID NO: 12) |
| hsa-miR-196a-3p | 3.315 | CGGCAACAAGAAACUGCCUGAG |
| | | (SEQ ID NO: 5) |
| hsa-miR-3195 | 2.845 | CGCGCCGGGCCCGGGUU |
| | | (SEQ ID NO: 1) |
| hsa-miR-32-5p | 1.74 | UAUUGCACAUUACUAAGUUGCA |
| | | (SEQ ID NO: 6) |
| hsa-miR-324-3p | 1.53 | ACUGCCCCAGGUGCUGCUGG |
| | | (SEQ ID NO: 22) |
| hsa-miR-326 | 1.7 | CCUCUGGGCCCUUCCUCCAG |
| | | (SEQ ID NO: 10) |
| hsa-miR-33a-5p | 2.07 | GUGCAUUGUAGUUGCAUUGCA |
| | | (SEQ ID NO: 18) |
| hsa-miR-3960 | 1.77 | GGCGGCGGCGGAGGCGGGGG |
| | | (SEQ ID NO: 19) |
| hsa-miR-4449 | 1.845 | CGUCCCGGGGCUGCGCGAGGCA |
| | | (SEQ ID NO: 14) |
| hsa-miR-4488 | 1.85 | AGGGGGCGGGCUCCGGCG |
| | | (SEQ ID NO: 9) |
| hsa-miR-4532 | 1.855 | CCCCGGGGAGCCCGGCG |
| | | (SEQ ID NO: 7) |
| hsa-miR-4792 | 1.45 | CGGUGAGCGCUCGCUGGC |
| | | (SEQ ID NO: 8) |
| hsa-miR-501-5p | 2.68 | AAUCCUUUGUCCCUGGGUGAGA |
| | | (SEQ ID NO: 15) |
| hsa-miR-629-5p | 1.975 | UGGGUUUACGUUGGGAGAACU |
| | | (SEQ ID NO: 20) |
| hsa-miR-7641 | 1.815 | UUGAUCUCGGAAGCUAAGC |
| | | (SEQ ID NO: 21) |
| hsa-miR-7704 | 1.91 | CGGGGUCGGCGGCGACGUG |
| | | (SEQ ID NO: 11) |

In addition, based on the same RNA-seq experiment, 12 specific miRNAs down-regulated in the inhibitory EVs originated from T47D shCtl (low invasive breast cancer cells naturally expressing NFAT3) compared to those produced in T47D shNFAT3 (in which the expression of NFAT3 is knocked down, i.e. cells that do not express NFAT3) were identified: hsa-miR-628-5p, hsa-miR-452-5p, hsa-miR-455-5p, hsa-miR-224-5p, hsa-miR-135a-5p, hsa-miR-99a-5p, hsa-miR-30c-5p, hsa-miR-30b-5p, hsa-let-7c-5p, hsa-miR-26a-5p, and hsa-miR-374b-5p.

### Identification of EV-miRNAs Regulating Cell Invasion/Motility

In order to identify, among the 21 miRNAs upregulated in SEVs of cells expressing NFAT3 (T47D shCtl), which ones were required for the SEVs to inhibit cell invasion/motility, the scratch wound assay was used in a 96 well plate with the Incucyte device. Indeed, in this assay, treatment with the inhibitory SEVs prevents the wound closure as it inhibits cell invasion.

Therefore, in a first approach, we choose to inhibit individually and independently each of the previously identified 21 miRNAs by transfecting MDA-MB-231 cells with antagomirs specific to each of the miRNAs and checking which ones were able to reverse the inhibitory effect of T47D shCtl SEVs in the scratch wound assay. Among the previously identified 21 miRNAs, we identified by antagomirs transfection 11 miRNAs that were actually involved in the anti-invasive effect of the SEVs (see Table 6 below).

In a second approach, we tested if individual and independent transfections of each of the previously identified 11 miRNAs alone in MDA-MB-231 cells were or not able to reproduce the anti-motility effect of T47D shCtl SEVs, but none of them were competent to do so (see Table 6 below).

TABLE 6
Effects on motility of the 21 miRNAs upregulated in T47D shCtl
SEVs compared to T47D shNFAT3 SEVs, tested either by transfecting
MDA-MB-231 cells with antagomirs specific to each of the miRNAs
and checking if the antagomir inhibits (+) or not(−)
the anti-motility effect of the T47D shCtl SEVs, or by transfecting
MDA-MB-231 cells independently with one of the miRNAs and checking
if the transfected miRNA alone reproduces (+) or not(−) the
anti-motility effect of the T47D shCtl SEVs.

Motility Validation Method

| 21 miRNAs upregulated in T47D | Antagomir | miRNA |
|---|---|---|
| shCtl SEVs compared to T47D | (inhibition | (inhibition |
| shNFAT3 SEVs | SEVs effect) | motility) |
| hsa-miR-195-5p | − | Not tested |
| hsa-miR-1277-3p | − | |
| hsa-miR-501-5p | − | |
| hsa-miR-3960 | − | |
| hsa-miR-7641 | − | |
| hsa-miR-33a-5p | − | |
| hsa-miR-629-5p | − | |
| hsa-miR-324-3p | − | |
| hsa-miR-188-5p | − | |
| hsa-miR-4449 | − | |
| hsa-miR-16-5p | + | |
| hsa-miR-196a-3p | + | − |
| hsa-miR-32-5p | + | − |
| hsa-miR-4488 | + | − |
| hsa-miR-326 | + | − |
| hsa-miR-4792 | + | − |
| hsa-miR-3195 | + | − |
| hsa-miR-1246 | + | − |
| hsa-miR-7704 | + | − |
| hsa-miR-188-3p | + | − |
| hsa-miR-4532 | + | − |

Based on the above results, we made the hypothesis that T47D shCtl SEVs might inhibit cell invasion/motility by the combined action of these 11 different miRNAs with the molar ratio shown in Table 7.

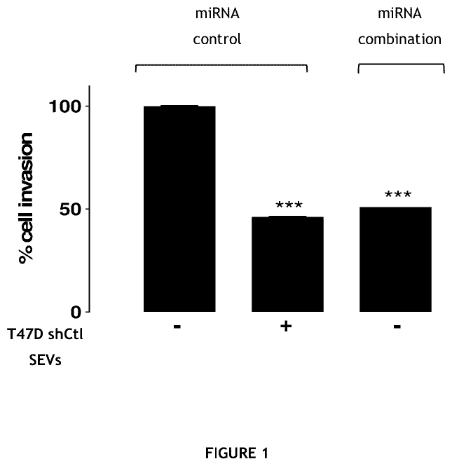

TABLE 7
Validated motility-inhibitory miRNAs. Fold increase: fold
increase of miRNA expression in T47D shCtl SEVs compared
to their average expression in T47D shNFAT3-3 and T47D
shNFAT3-4 SEVs. Ratio miRNA: ratio calculated with miRNA
196a-3p set at 1. This ratio for each miRNA was followed
in the miRNAs combination used in FIG. 1.

| No | miR | Fold increase | ratio |
|---|---|---|---|
| 1 | hsa-miR-3195 | 2.8 | 0.9 |
| 2 | hsa-miR-1246 | 2.1 | 0.6 |
| 3 | hsa-miR-188-3p | 2.1 | 0.6 |
| 4 | hsa-miR-16-5p | 2.0 | 0.6 |
| 5 | hsa-miR-196a-3p | 3.3 | 1.0 |
| 6 | hsa-miR-32-5p | 1.7 | 0.5 |
| 7 | hsa-miR-4532 | 1.9 | 0.6 |
| 8 | hsa-miR-4792 | 1.5 | 0.4 |
| 9 | hsa-miR-4488 | 1.9 | 0.6 |
| 10 | hsa-miR-326 | 1.7 | 0.5 |
| 11 | hsa-miR-7704 | 1.9 | 0.6 |

Indeed, we validated that transfecting this combination of the 11 miRNAs was sufficient to reproduce the same inhibitory effect on cell invasion as T47D shCtl SEVs did in the MDA-MB-231 cell line (see FIG. 1).

### Identification of EV-miRNAs Regulating Hetero Spheroids Growth

At the time of the filing of WO2017167788A1, we were not able to explain how the SEVs or NFAT3-expressing cells hindered tumor growth in vivo. Since SEVs are known to be powerful cell signaling providers on both tumoral cells and the tumoral microenvironment, we hypothesized that the injected SEVs in our breast cancer mice model might induce phenotypic modifications not only in cancer cells but also, directly or indirectly in the cells presents in the tumoral microenvironment. These cells present in the tumoral microenvironment, such as cancer associated fibroblasts and macrophages, can have a direct impact on tumor growth (Cook, J. [symbol] Hagemann, T. Tumour-associated macrophages and cancer. Curr Opin Pharmacol 13, 595-601 (2013)).

Based on previous findings showing that infiltrating macrophages were effectively present within the orthotopic tumor (see FIG. 3B of de Camargo, L. C. B et al. Sci Rep 10, 8964 (2020)), SEVs from NFAT3-expressing cells did not inhibit the growth of cancer cells spheroids in the absence of macrophages (see FIG. 3A of de Camargo, L. C. B et al. Sci Rep 10, 8964 (2020)) but inhibited the growth of cancer cells/macrophages hetero-spheroids, inhibition that was associated with an increase of apoptosis (see FIG. 3C of de Camargo, L. C. B et al. Sci Rep 10, 8964 (2020)).

Thanks to this in vitro hetero-spheroids growth test, we tested among the previously identified 21 miRNAs upregulated in T47D shCtl SEVs compared to T47D shNFAT3 SEVs which ones were required for the T47D shCtl SEVs to inhibit hetero-spheroids growth. As in the case of motility assessment, the effect on hetero-spheroids growth was tested:
firstly by transfecting MDA-MB-231 cells with antagomirs specific to each of the miRNAs and checking effect on each antagomir on inhibition of hetero-spheroids growth, and
secondly by transfecting MDA-MB-231 cells independently with one of the miRNAs and checking if the transfected miRNA alone reproduces (+) or not (−) the inhibitory effect of the T47D shCtl SEVs on hetero-spheroids growth.

Results are presented in Table 8 below and show that:
miRNAs are required for the T47D shCtl SEVs to inhibit hetero spheroids growth in vitro, since antagomir transfection of each of these 8 miRNAs inhibit the inhibitory effect of T47D shCtl SEVs: hsa-miR-195-5p, hsa-miR-501-5p, hsa-miR-324-3p, hsa-miR-4449, hsa-miR-16-5p, hsa-miR-196a-3p, hsa-miR-4488, and hsa-miR-7704, and
Contrary to the miRNAs required to inhibit the cell motility, 7 of the 8 miRNAs (all excepthsa-miR-501-5p) found to be involved in inhibition of hetero spheroids growth using the antagomir approach were able alone to inhibit hetero spheroid growth.

TABLE 8
Effects on hetero-spheroids growth of the 21 miRNAs upregulated
in T47D shCtl SEVs compared to T47D shNFAT3 SEVs, tested either
by transfecting MDA-MB-231 cells with antagomirs specific to
each of the miRNAs and checking if the antagomir inhibits (+) or
not(−) the inhibitory effect of the T47D shCtl SEVs on hetero-
spheroids growth, or by transfecting MDA-MB-231 cells independently
with one of the miRNAs and checking if the transfected miRNA
alone reproduces (+) or not(−) the inhibitory effect of
the T47D shCtl SEVs on hetero-spheroids growth.

| | Hetero spheroids growth validation method | |
|---|---|---|
| | Antagomir | miRNA |
| miRNAs up-regulated in | (inhibition of | (inhibition of |
| T47D shCtl SEV compared | T47D shCtl | hetero- |
| to T47D shNFAT3 SEVs | SEVs effect) | spheroids growth) |
| hsa-miR-195-5p | + | + |
| hsa-miR-1277-3p | − | Not tested |
| hsa-miR-501-5p | + | − |
| hsa-miR-3960 | − | Not tested |
| hsa-miR-7641 | − | Not tested |
| hsa-miR-33a-5p | − | Not tested |
| hsa-miR-629-5p | − | Not tested |
| hsa-miR-324-3p | + | + |
| hsa-miR-188-5p | − | Not tested |
| hsa-miR-4449 | + | + |
| hsa-miR-16-5p | + | + |
| hsa-miR-196a-3p | + | + |
| hsa-miR-32-5p | − | Not tested |
| hsa-miR-4488 | + | + |
| hsa-miR-326 | − | Not tested |
| hsa-miR-4792 | + | + |
| hsa-miR-3195 | − | Not tested |
| hsa-miR-1246 | − | Not tested |
| hsa-miR-7704 | + | + |
| hsa-miR-188-3p | − | Not tested |
| hsa-miR-4532 | − | Not tested |

In addition, it should be noted that 4 of the 8 miRNAs involved in the inhibitory effect on hetero-spheroids growth of T47D shCtl SEVs belong to the 11 miRNAs combination found to inhibit motility: hsa-miR-16-5p, hsa-miR-196a-3p, hsa-miR-4488, and hsa-miR-7704.

### Conclusions

All in all, our results have identified:
a critical combination of 11 miRNAs present within the inhibitory T47D shCtl SEVs sufficient to inhibit in vitro cell motility, and
8 miRNAs that are involved in the inhibitory effect of T47D shCtl SEVs on the growth of hetero spheroids, 7 of which are able independently to inhibit hetero spheroids growth.

On this basis, an anticancer treatment comprising the 11 miRNAs combination should be able to inhibit both cancer growth and cancer spreading (metastasis). Adding at least one of the 4 miRNAs able independently to inhibit hetero spheroids growth that are not in the 11 miRNAs combination may further inhibit cancer growth.

### Example 2: Confirmation of the Anticancerous Effect of a miRNA Composition Comprising 15 miRNAs (the Critical Combination of 11 miRNAs and the 4 Additional miRNAs Able Independently to Inhibit Hetero Spheroids Growth that are not in the 11 miRNAs Combination)

The inventors further validated that the transfection of the combination of the 15 miRNAs identified (miRNA Comb) at 50nM was able to fully reproduce the inhibitory effect of the SEVs described in Camargo et al., 2020 in 2 triple negative breast cancer cell lines (MDA-MB-231 and SUM-159PT) compared to the same cells transfected with a control miRNA.

### Materials and Methods

miRNA Composition and Control miRNA

The composition of miRNA Comb is presented in Table 9 below:

TABLE 9
List of the 15 miRNAs, functionally validated, up-regulated in
SEVs produced by T-47D shCtl cells compared to the miRNAs present
in SEVs produced by T-47D shNFAT3 cells and included in the
tested composition of 15 miRNA (miRNA Comb). Ratio: for each
miRNA, corresponds to the ratio calculated with miRNA 195-5p
set at 1 obtained from the RNAseq experiments depicting the
relative quantity of each miRNA compared to the miRNA 195-5p
in the inhibitory SEVs produced by T-47D shCtl cells.

| | Anti-invasive | |
|---|---|---|
| | AND | Anti-proliferative |
| Anti-invasive miRNA | Anti-proliferative miRNA | miRNA |
| (ratio) | (ratio) | (ratio) |
| hsa-miR-4532 | hsa-miR-188.3p | hsa-miR-195-5p |
| (0.50) | (0.57) | (1) |
| hsa-miR-4792 | hsa-miR-16-5p | hsa-miR-501-5p |
| (0.39) | (0.55) | (0.71) |
| hsa-miR-326 | hsa-miR-4488 | hsa-miR-324-3p |
| (0.46) | (0.50) | (0.41) |
| hsa-miR-7704 | hsa-miR-196a-3p | hsa-miR-4449 |
| (0.51) | (0.89) | (0.49) |
| hsa-miR-3195 | | |
| (0.76) | | |
| hsa-miR-1246 | | |
| (0.57) | | |
| hsa-miR-32-5p | | |
| (0.47) | | |

### Cell Transfection

2 triple negative breast cancer cell lines (MDA-MB-231 and SUM-159PT) have been transfected either with tested composition of 15 miRNA (miRNA Comb) or with a control miRNA (miRNA Ctrl), using the same protocol as in Example 1.

### Hetero-Spheroid Growth Assay and Apoptosis Detection

The same protocol as in Example 1 was used.

### Scratch Wound Assay

The same protocol as in Example 1 was used.

### Results and Conclusion

Results are presented in FIG. 2. Similarly to SEVs produced by T-47D shCtl cells, the tested 15 miRNAs composition reduces growth (FIG. 2A) and invasion (FIG. 2C) of 2 triple negative breast cancer cell lines (MDA-MB-231 and SUM-159PT), and increase their apoptosis (FIG. 2B).

These results confirm the very good anticancer properties of the tested 15 miRNAs composition.

1. A miRNA composition comprising the following isolated 11 miRNAs: hsa-miR-3195, hsa-miR-1246, hsa-miR-188-3p, hsa-miR-16-5p, hsa-miR-196a-3p, hsa-miR-32-5p, hsa-miR-4532, hsa-miR-4792, hsa-miR-4488, hsa-miR-326, and hsa-miR-7704.

2. The miRNA composition according to claim 1, wherein the 11 miRNAs are present in the composition in the following relative ratios:

| | Relative ratio in |
|---|---|
| miRNA | the miRNA composition |
| hsa-miR-3195 | 0.8 to 1.0 |
| hsa-miR-1246 | 0.5 to 0.7 |
| hsa-miR-188-3p | 0.5 to 0.7 |
| hsa-miR-16-5p | 0.5 to 0.7 |
| hsa-miR-196a-3p | 1.0 |
| hsa-miR-32-5p | 0.4 to 0.6 |
| hsa-miR-4532 | 0.5 to 0.7 |
| hsa-miR-4792 | 0.3 to 0.5 |
| hsa-miR-4488 | 0.5 to 0.7 |
| hsa-miR-326 | 0.4 to 0.6 |
| hsa-miR-7704 | 0.5 to 0.7 |

wherein the relative ratio of each miRNA in the composition is calculated with the ratio of hsa-miR-196a-3p set at 1.

3. The miRNA composition according to claim 1, which further comprises at least one miRNA selected from hsa-miR-195-5p, hsa-miR-324-3p, hsa-miR-4449 and hsa-miR-501-5p.

4. The miRNA composition according to claim 1, wherein said composition comprises at most 50 distinct miRNAs.

5. A delivery vector loaded with the miRNA composition according to claim 1.

6. The delivery vector of claim 5, which is a liposome loaded with the miRNA composition.

7. The delivery vector of claim 5, which is a SEV loaded with the miRNA composition.

8. A method for preparing the loaded SEVs according to claim 7, comprising:
a) preparing SEVs from healthy cells,
b) loading the SEVs with the miRNA composition.

9. The method according to claim 8, wherein the loading of the SEVs involves:
pH gradient modification of the SEVs, or
creating a turbulent flow in the liquid medium containing:
the SEVs, wherein the Kolmogorov length of the flow is less than or equal to 100 μm, or
the cells producing the SEVs, wherein the Kolmogorov length of the flow is less than or equal to 50 μm.

10. A pharmaceutical composition comprising the miRNA composition according to claim 1.

11. (canceled)

12. A method of treating cancer comprising administering to a subject in need thereof a pharmaceutical composition according to claim 10.

13. The method of treating cancer according to claim 12, wherein said cancer is selected from solid cancers.

14. The method of treating cancer according to claim 12, wherein said cancer is a metastatic cancer.

15. The method of treating cancer according to claim 12, wherein said pharmaceutical composition is intended to be administered by intravenous or intratumoral route.

16. The method of treating cancer according to claim 13, wherein the cancer is selected from breast cancer, melanoma, pancreatic cancer, glioblastoma, colorectal cancer, and lung cancer. cancer.

17. The method of treating cancer according to claim 13, wherein the cancer is breast

18. A pharmaceutical composition comprising the delivery vector of claim 5.

19. A pharmaceutical composition comprising the SEV prepared according to the method of claim 8.


SEQUENCE LISTING (22 sequences)

SEQ ID NO: 1 - RNA, 17 nt, Homo sapiens
  misc_feature: hsa-miR-3195
cgcgccgggcccggguu

SEQ ID NO: 2 - RNA, 19 nt, Homo sapiens
  misc_feature: hsa-miR-1246
aauggauuuuuggagcagg

SEQ ID NO: 3 - RNA, 21 nt, Homo sapiens
  misc_feature: hsa-miR-188-3p
cucccacaugcaggguuugca

SEQ ID NO: 4 - RNA, 22 nt, Homo sapiens
  misc_feature: hsa-miR-16-5p
uagcagcacguaaauauuggcg

SEQ ID NO: 5 - RNA, 22 nt, Homo sapiens
  misc_feature: hsa-miR-196a-3p
cggcaacaagaaacugccugag

SEQ ID NO: 6 - RNA, 22 nt, Homo sapiens
  misc_feature: hsa-miR-32-5p
uauugcacauuacuaaguugca

SEQ ID NO: 7 - RNA, 17 nt, Homo sapiens
  misc_feature: hsa-miR-4532
ccccggggagcccggcg

SEQ ID NO: 8 - RNA, 18 nt, Homo sapiens
  misc_feature: hsa-miR-4792
cggugagcgcucgcuggc

SEQ ID NO: 9 - RNA, 18 nt, Homo sapiens
  misc_feature: hsa-miR-4488
agggggcgggcuccggcg

SEQ ID NO: 10 - RNA, 20 nt, Homo sapiens
  misc_feature: hsa-miR-326
ccucugggcccuuccuccag

SEQ ID NO: 11 - RNA, 19 nt, Homo sapiens
  misc_feature: hsa-miR-7704
cggggucggcggcgacgug

SEQ ID NO: 12 - RNA, 21 nt, Homo sapiens
  misc_feature: hsa-miR-195-5p
uagcagcacagaaauauuggc

SEQ ID NO: 13 - RNA, 23 nt, Homo sapiens
  misc_feature: hsa-miR-324-3p first sequence
cccacugccccaggugcugcugg

SEQ ID NO: 14 - RNA, 22 nt, Homo sapiens
  misc_feature: hsa-miR-4449
cgucccggggcugcgcgaggca

SEQ ID NO: 15 - RNA, 22 nt, Homo sapiens
  misc_feature: hsa-miR-501-5p
aauccuuugucccugggugaga

SEQ ID NO: 16 - RNA, 22 nt, Homo sapiens
  misc_feature: hsa-miR-1277-3p
uacguagauauauauguauuuu

SEQ ID NO: 17 - RNA, 21 nt, Homo sapiens
  misc_feature: hsa-miR-188-5p
caucccuugcaugguggaggg

SEQ ID NO: 18 - RNA, 21 nt, Homo sapiens
  misc_feature: hsa-miR-33a-5p
gugcauuguaguugcauugca

SEQ ID NO: 19 - RNA, 20 nt, Homo sapiens
  misc_feature: hsa-miR-3960
ggcggcggcggaggcggggg

SEQ ID NO: 20 - RNA, 21 nt, Homo sapiens
  misc_feature: hsa-miR-629-5p
uggguuuacguugggagaacu

SEQ ID NO: 21 - RNA, 19 nt, Homo sapiens
  misc_feature: hsa-miR-7641
uugaucucggaagcuaagc

SEQ ID NO: 22 - RNA, 20 nt, Homo sapiens
  misc_feature: hsa-miR-324-3p second sequence
acugccccaggugcugcugg